(19) United States
(12) Patent Application Publication
(10) Pub. No.: US 2003/0098027 A1
(43) Pub. Date: May 29, 2003

(54) Belt and bending attachment for belt

(76) Inventor: Syuji Mori, Tsukuba-gun (JP)

Correspondence Address: Syuji Mori, 98-12 Aoki, Inamachi, Tsukuba-gun, Ibaraki-ken, 300-2346, JP

(21) Appl. No.: 09/995,164

(22) Filed: Nov. 29, 2001

(51) Int. Cl. A61F 5/37

(52) U.S. Cl. 128/876

(57) ABSTRACT

A belt, inducing abdominal breathing, comprises a belt cloth; an attachment provided at one end portion of the belt cloth; a buckle attached in an end of another side of the belt cloth so that it may engage with the attachment, regulating the length of the belt cloth, fixing the belt cloth and pressing upper part of the pubis; a belt bending attachments slidely and detachably provided to the belt cloth, capable of bending the belt cloth. Therefore it fastens hypogastric region efficiently so that it can be enriched energy, and that fastens easily bound tight in the optimal state with one belt and can be bound tight in the optimal state without spoiling a design.

### BACKGROUND OF THE INVENTION

The present invention relates to a belt and belt bending attachment that fastens the waist, pelvis, upper part of pubis at hypogastric region, and pressurizes along with upper part of pubis and inguinal region, and abdominal breathing is urged at the same time

Conventionally, Japanese people are wearing a belt of Japanese clothes, geestring (FUNDOSHI), etc., peculiar to Japan.

In the conventional belt and geestring of Japanese clothes, when it considered the physiological function in a human body, it should not leave. From ancient times in Japan, there are lots of phrases related to belly which is located at the portion applied to the pubis upper part from under a navel, such as “a person with guts”, “a belly being thick”, and “a belly being made”, and by fastening hypogastric region by the belt, geestring, etc., abdominal pressure goes up, abdominal breathing become free and it has formed the person full of energy, and self-possessed. However, in modern society, since it becomes clothes and a belt is generally fastened on a navel by trousers, he does not necessarily fasten hypogastric region, and it is considered that people forget abdominal breathing and become quite weak physiologically and mentally. Therefore energy does not come out in the state with weak hypogastric region. On the contrary, hypogastric region of human beings such as a mentally deranged person and a neurosis must be whether it has become stringy and flabby muscle.

### SUMMARY OF THE INVENTION

Accordingly, it is an object of the present invention to provide a belt and belt bending attachment that fastens hypogastric region efficiently so that it can be enriched energy, and that fastens easily bound tight in the optimal state with one belt,,

In addition, it is an object of the present invention to provide a belt and belt bending attachment that can be bound tight in the optimal state without spoiling a design.

The novel features which are believed to be characteristic of the invention, both as to its organization and method of operation, together with further objects and advantages thereof, are described below with reference to the accompanying drawings in which a presently preferred embodiment of the invention is illustrated as an example.

It is to be expressly understood, however, that the drawings are for the purpose of illustration and description only, and are not intended as a definition of the limits of the invention.

### BRIEF DESCRIPTION OF THE DRAWINGS

FIG. 1 is an explanation view of a belt showing a first embodiment of the present invention;

FIG. 2 is an explanation view of a belt showing a first embodiment of the present invention;

FIG. 3 is an explanation view of a belt of the way in which the belt is installed;

FIG. 4 is an explanation view of a buckle showing the first embodiment of the present invention;

FIG. 5 is an explanation view of a buckle showing the first embodiment of the present invention;

FIG. 6 is an explanation view of a belt bending attachment showing the first embodiment of the present invention;

FIG. 7 is an explanation view showing how to pass a belt through the belt bending attachment;

FIG. 8 is a front view of an ornament of a belt bending attachment showing the first embodiment of the present invention;

FIG. 9 is a top view of an ornament of a belt bending attachment showing the first embodiment of the present invention

FIG. 10 is a right side view of an ornament of a belt bending attachment showing the first embodiment of the present invention

FIG. 11 is a sectional view taken along a line 11-11 of FIG. 10

FIG. 12 is an explanation view showing the way in which the belt passes through the belt bending attachment;

FIG. 13 is an explanation view showing the way in which the belt attached to the belt bending attachment;

FIG. 14 is an explanation view showing the way in which the belt is passed through the belt bending attachment;

FIG. 15 is an explanation view showing the way in which the belt is wearing;

FIG. 16 is an explanation view showing the way in which the belt is wore;

FIG. 17 is an explanation view when in use;

FIG. 18 is another explanation view when in use;

FIG. 19 is a top view of a buckle showing a second embodiment of the present invention;

FIG. 20 is a front view of a buckle showing the second embodiment of the present invention;

FIG. 21 is a bottom view of a buckle showing the second embodiment of the present invention;

FIG. 22 is a right side view of a buckle showing the second embodiment of the present invention;

FIG. 23 is an explanation view of a buckle body showing the second embodiment of the present invention

FIG. 24 is a sectional view taken along a line 24-24 of FIG. 23;

FIG. 25 is an explanation view of a clasp showing the second embodiment of the present invention;

FIG. 26 is an explanation view showing the way in which the belt is wore;

FIG. 27 is an explanation view of a belt bending attachment showing a third embodiment of the present invention;

FIG. 28 is a front view of a belt bending attachment showing the third embodiment of the present invention;

FIG. 29 is a top view of a belt bending attachment showing the third embodiment of the present invention;

FIG. 30 is a right side view of a belt bending attachment showing the third embodiment of the present invention; and

FIG. 31 is a sectional view taken along a line 31-31 of FIG. 29.

### DETAILED DESCRIPTION OF THE PREFERRED EMBODIMENTS

Preferred embodiments of the present invention are described in more detail below referring to the accompanying drawings.

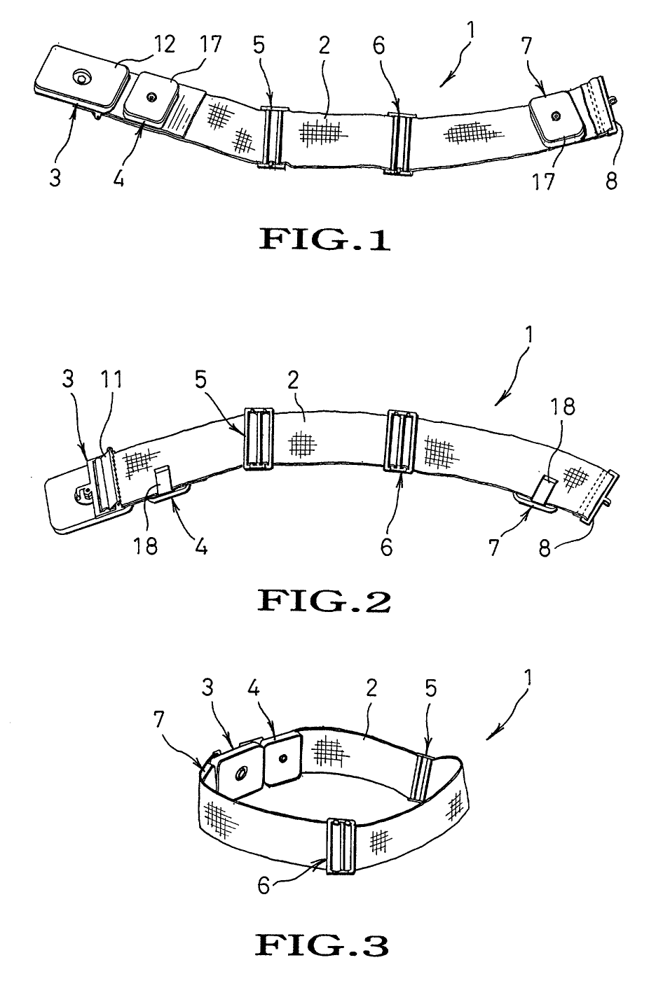

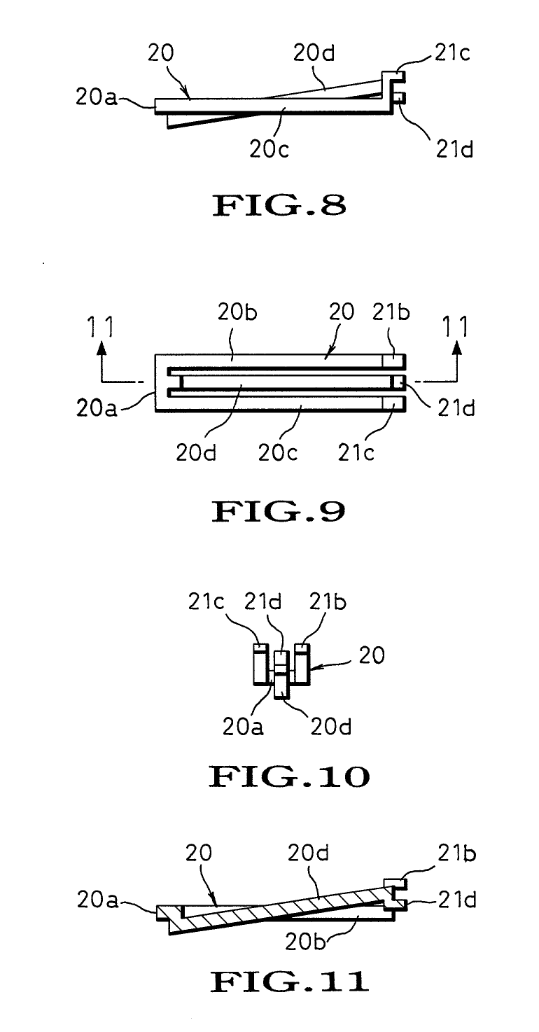

FIGS. 1 to 18 illustrate a belt of a first embodiment of the present invention. As illustrated in FIG. 1 or FIG. 3, the belt 1 comprises a belt cloth 2, a buckle 3 attached in one end of the belt cloth 2, an inguinal region pressurization implement 4 which fixed steadily attached in part adjacent to the buckle 3 of the belt cloth 2, a belt bending attachments 5 and 6 attached in the belt cloth 2, an inguinal region pressurization implement 7 of another side attached fixedly in the end slippage part of another side of the belt cloth 2, and a clasp 8 attached fixedly in the end part at another side of the belt cloth 2.

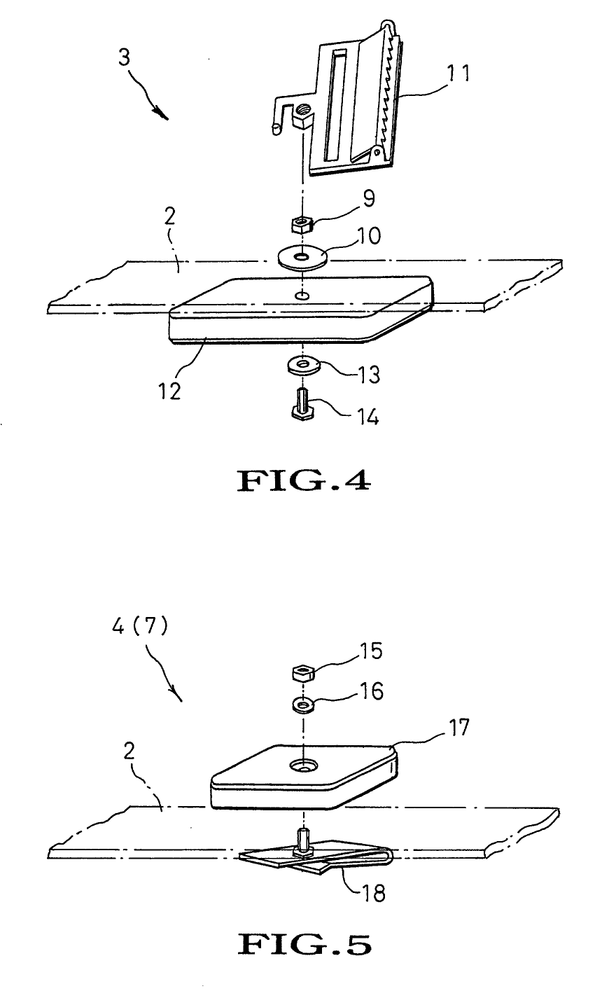

The belt cloth 2 is a cloth including the same product made of cloth as usual, and its width is suitable for 5 to 6 cm preferably. If the width is too wide or it is elastic, the tightness condition of hypogastric region will not be good. The buckle 3 is used as a component which applies pressure to the upper part of the pubis, and as illustrated in FIG. 4, the belt length adjustment ornament 11 is located in the back of one end of the belt cloth 2 through a nut 9 and a washer 10, and a bolt 14 is inserted through the rubber board 12 and a washer 13 from the surface side of the belt cloth 2, and the bolt 14 combines with the screw part of the belt length adjustment ornament 11 and fixed integrally.

In addition, when a belt equips with this belt, the side that touches a human body means a backside, and the surface of a belt means the opposite one.

As illustrated in FIG. 5, in the inguinal region pressurization implements 4 and 7, a nut 15 and a washer 16 are embedded in the rubber board 17, and a clip 18 they are attached from the surface part of the belt cloth 2 so that it may be united with the nut 15, washer 16, rubber board 17, and belt cloth 2.

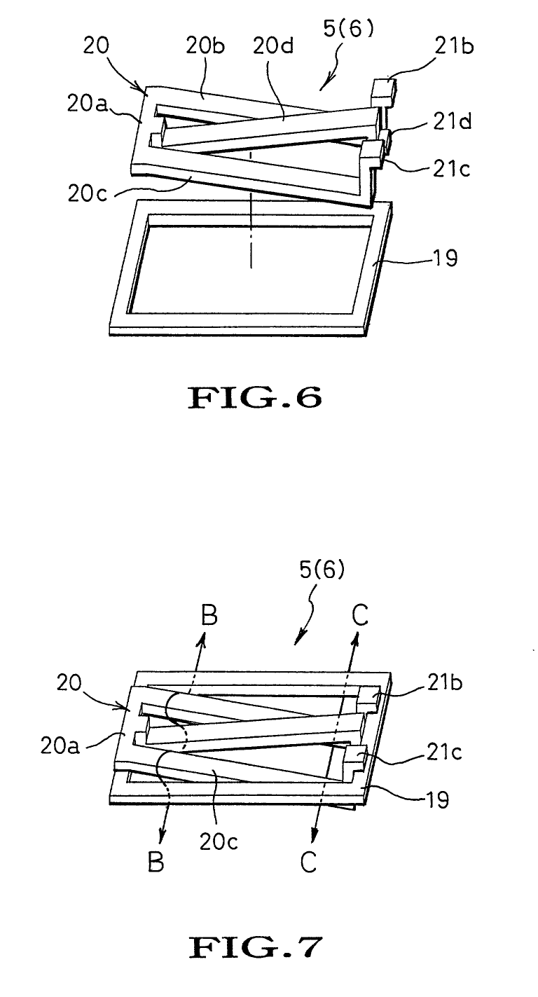

As illustrated in FIG. 6 to FIG. 11, the belt bending attachments 5 and 6 include frame ornament 19 formed in the shape of a square and an ornament 20 formed in the shape of an E letter which can be bent the belt cloth 2 in the state that the belt cloth 2 put between the frame ornament 19. The ornament 20 further includes a root part 20a, clip sticks 20b and 20c formed so that they project perpendicular in the horizontal direction from both ends of the root part 20a, having the tip part thereof formed in the shape of a crank, and a central clip stick 20d formed so that it projects perpendicular from a central part of the root part 20a by bending the attached part to the root part 20a downwardly, having a tip part thereof formed in reverse crank form comparing with that of the clip sticks 20b and 20.

Furthermore, as illustrated in FIG. 7, it shows how to pass by B-B and C-C directions along belt cloth. The portion along which the B-B direction passes, that is near the root part 20a of the ornament 20 bends wavy, and the C-C direction passes along near the tip parts 21b, 21d and 21c of the ornament 20 passes approximately level. The difference of bending of the B-B direction and the C-C direction makes a bending of the belt cloth 2.

Then one side located in the length direction of the frame ornament 19 can be put by the tip parts 21b and 21c and tip part 21d having a reverse crank shape of the ornament 20a.

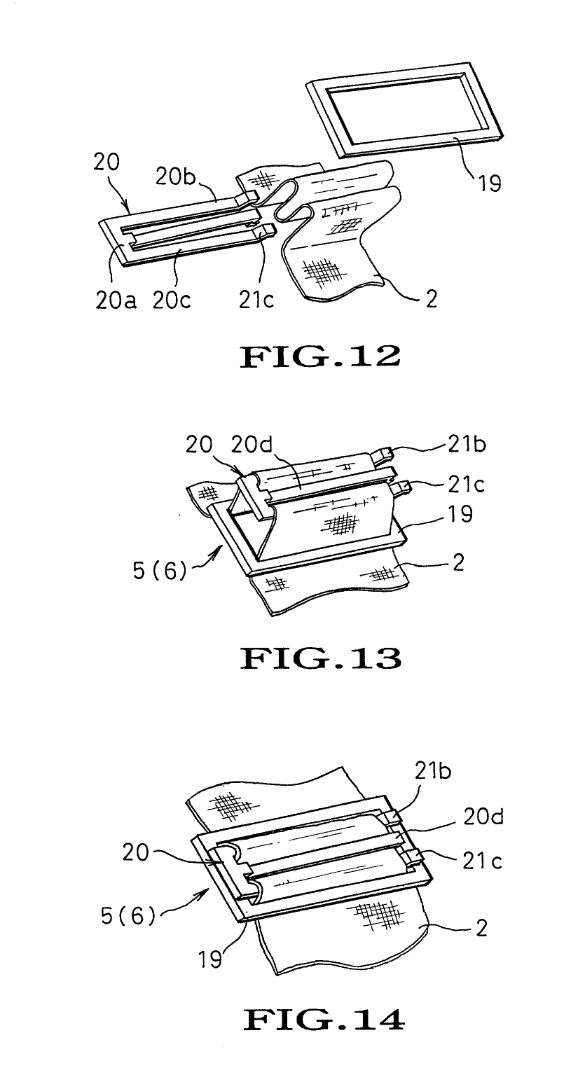

As illustrated in FIG. 12 to FIG. 14, in the belt 1 of the present invention, the belt cloth 2 is given bending and forms two mountains and one valley, and the clip sticks 20b and 20c of ornament 20 pass into the portion of the two mountains, and the central clip stick 20d passes into the portion of the valley. Next, as illustrated in FIG. 13, after the ornament 20 which put the belt cloth 2 is passed the frame ornament 19, one side of the length direction of the frame ornament 19 is clipped by the tip parts 21b, 21c, and 21d of the clip stick 20 of the ornament 20. After that, when the both end of the belt cloth which is passed through the frame ornament 19 is pulled, the bending of belt cloth will be completed as illustrated in FIG. 14.

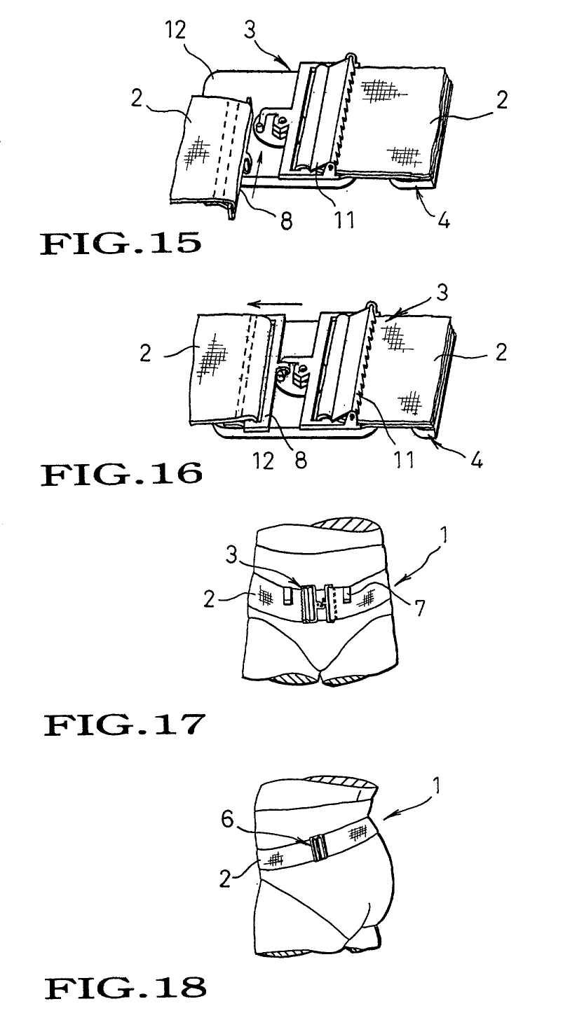

Next, as illustrated in FIG. 15 to FIG. 18, the clasp 8 is sewn by the sewing machine etc. at the end of another side of the belt cloth 2, an by the belt length adjustment ornament 11, the length from a belt user's waist to inguinal region and the pubis upper part is decided, and it fixes to one end. Then, if moderate pressure is applied not only to the pubis upper part but to the whole inguinal region when the inguinal region and the pubis upper part are equipped with a belt from the waist so that it becomes the condition which has held the whole hypogastric region, and pressure may be applied also to the inguinal region besides the buckle 3. Since it has noticed coming to be able to do abdominal breathing more comfortably, the attachment clip 18 of the inguinal region pressurization implements 2 and 7 is used the inguinal region pressurization implements 4 and 7 for the suitable portion of the right and left inguinal region of, and it attaches in the back of the belt.

When the belt is turned around the inguinal region and the upper part of the pubis and is fastened from the waist, since the bending arises according to that the surface of a human body is continuation of a curved surface and the height difference of the waist and the pubis upper part, the belt bending attachments 5 and 6 attach a little behind the both sides of the waist for canceling the bending.

In addition, the thickness of the rubber boards 13 and 17 has less than 1 cm, and the width is suitable to the extent that it is the same as the width of the belt preferably.

Moreover, although the rubber board is used in this embodiment, you may use not only it but also the elasticity material and shock absorption material, which have hardness to some extent.

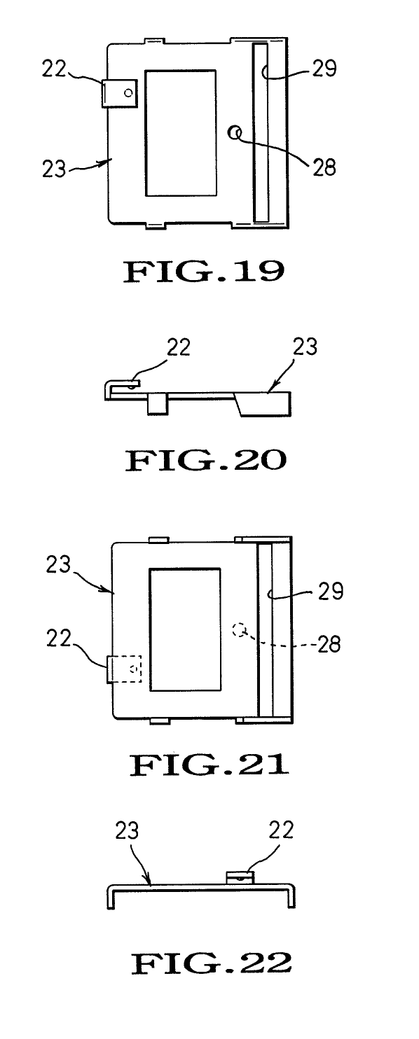

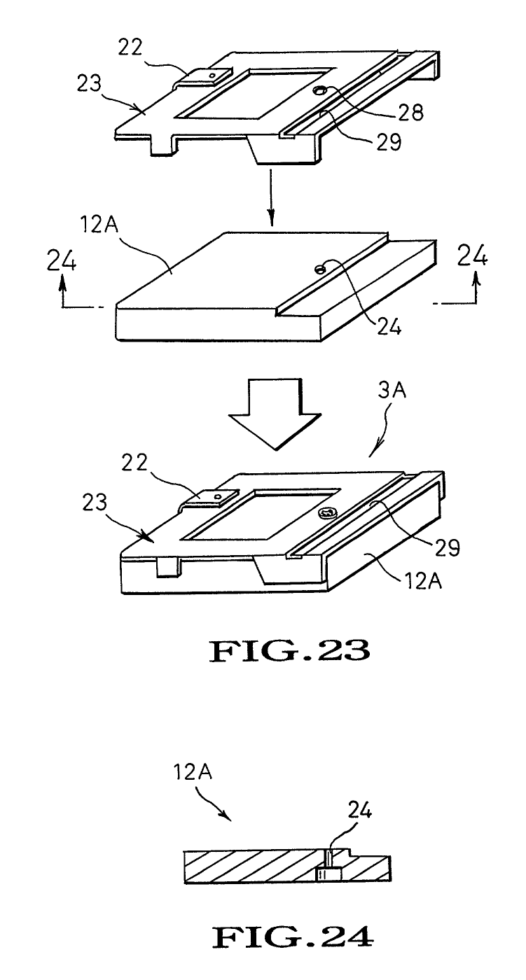

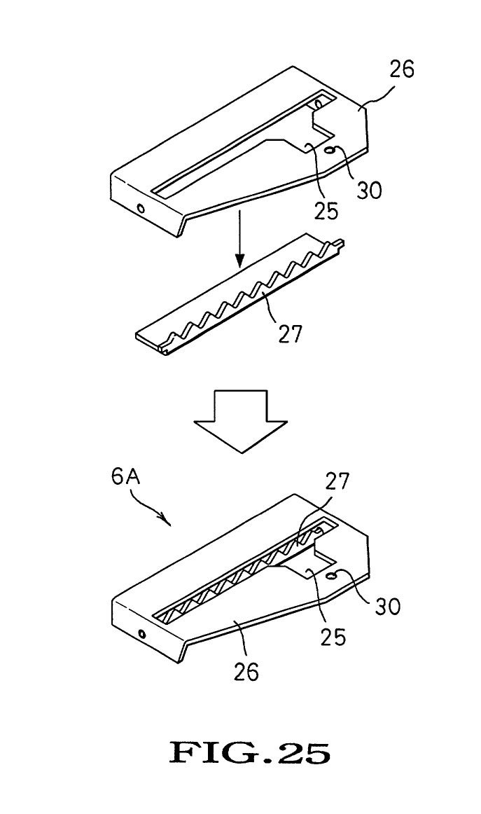

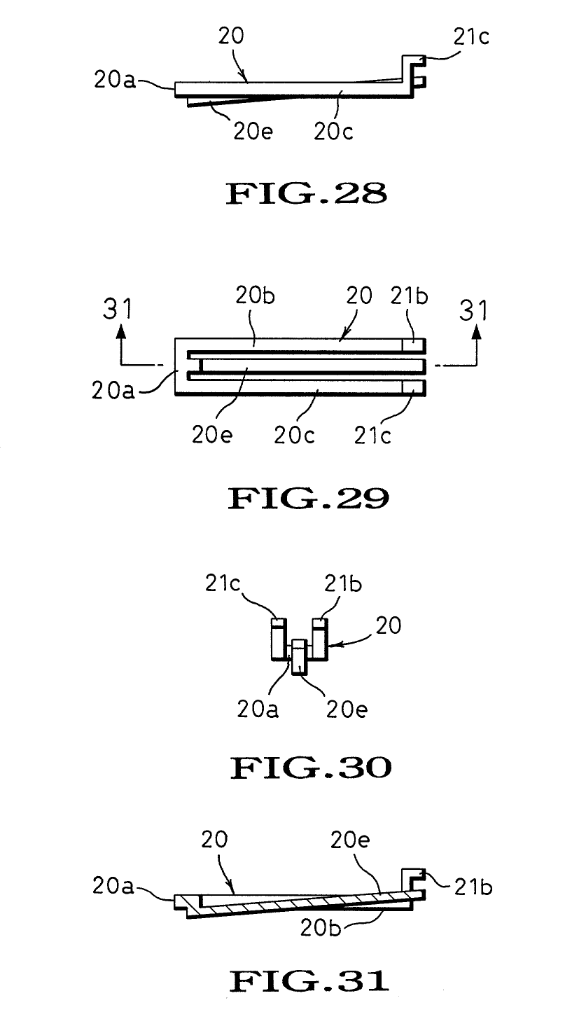

Other embodiments of the present invention will now be described referring to FIGS. 19 to 31. Through the drawings of the embodiments, like components are denoted by like numerals as of the first embodiment and will not be further explained in great detail.

A second embodiment of the present invention is illustrated in FIGS. 19 to 26. It is distinguished from the first embodiment by the fact that the buckle 3 is replaced with another buckle 3A, and the clasp 8 is replaced with another clasp 8A. The buckle 3A further includes a rubber board 12A having a buckle body 23 made with metal with a hook 22 formed in the shape of a L letter and a screw insertion hole 24 attached with the buckle body 23 integrally. The clasp 8A further include a clasp body 26 having an engage hole 26 which can hook on the hook 22 and an insertion hole 30 inserted a screw, nut etc. thereinto and an engage part 27 engaged so as to adjust a length by hooking the belt cloth 2 on the both ends of the clasp body 26

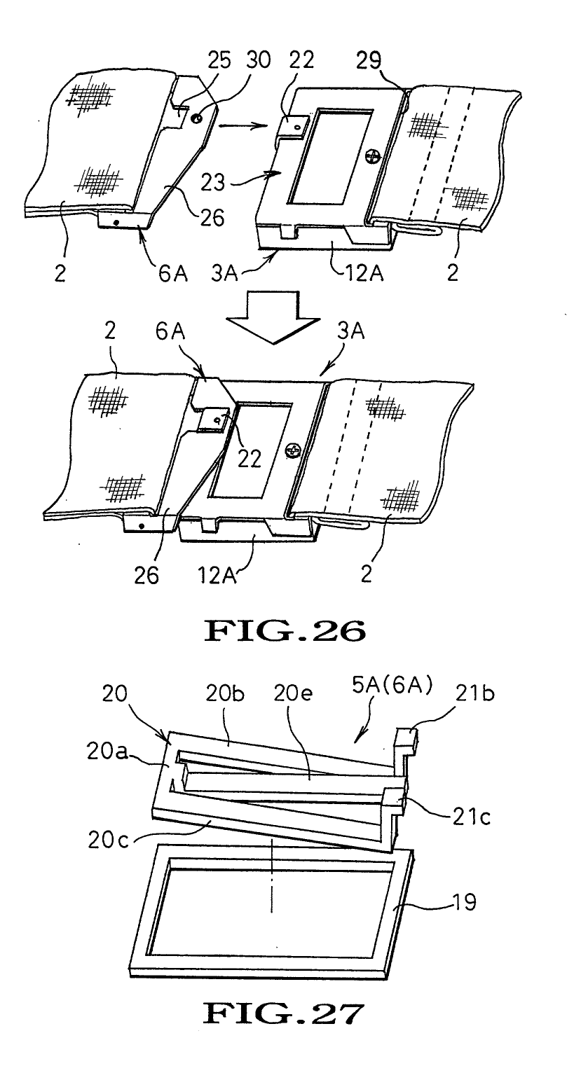

A belt 1A according to the second embodiment has similar advantages to that according to the first embodiment and it can fasten firmly as illustrated in FIG. 26.

A third embodiment of the present invention is illustrated in FIGS. 27 to 31. It is distinguished from the first embodiment by the fact that the belt bending attachments 5A and 6A using the ornament 20A formed in the shape of a E letter which has a central clip stick 20e prepared so as to project nearly perpendicularly from the central part of the root part 20a is used. A belt 1B with the belt bending attachments 5A and 6A according to the third embodiment has similar advantages to that according to the first embodiment.

In addition, although each embodiment in the present this invention differs mainly explained based on the first embodiment, even if it uses this invention combining the composition used not only the elements of the first embodiment but that of each embodiment, the same action effect is acquired.

As set forth above, the advantages of the invention are as follows:

(1) A belt, inducing abdominal breathing, comprises a belt cloth; an attachment provided at one end portion of the belt cloth; a buckle attached in an end of another side of the belt cloth so that it may engage with the attachment, regulating the length of the belt cloth, fixing the belt cloth and pressing upper part of the pubis; a belt bending attachments slidely and detachably provided to the belt cloth, capable of bending the belt cloth so that a belt can be turned to inguinal region and the pubis upper part and from the waist and can be fastened, pressure can be further applied to the pubis upper part and inguinal region efficiently, and involuntary abdominal breathing can be made to maintain. That is, when it is in the state of breathing when the belt is fastened, since the belt is fastened along with inguinal region in the state that the rise of internal pressure of abdomen in the time of natural breathing and an abdomen spread outside, work in the direction where an abdomen spreads more rather than suppressing an abdomen, and a diaphragm is lowered at the same time the power in the further abdomen fastens hypogastric region, and the amount of breathing comes together and increases.

(2) As discussed above, by fastening a belt, it is started by abdominal pressure, and it can bring about strain good for the muscles and internal organs inside abdomen and can make it activated.

(3) As discussed above, while a feeling can settle down calmly and is carrying out calmly, a feeling of fullness is not lost. Since it can respond to mind at an abdomen even when exposed to various stress, irritation and the degree that carries out potassium decrease very much. Furthermore, it is deep, and for a long time, quiet abdominal breathing can suppress easily that self-consciousness riots and turns, and can bring about a sense of togetherness with nature efficiently.

(4) As discussed above, since it has a simple structure, it can be carried out easily, it can manufacture cheaply.

What is claimed is:

1. A belt, inducing abdominal breathing, comprising:
a belt cloth;
an attachment provided at one end portion of the belt cloth;
a buckle attached in an end of another side of the belt cloth so as to engage with the attachment, regulating the length of the belt cloth, fixing the belt cloth and pressing upper part of the pubis; and
a belt bending attachments slidely and detachably provided to the belt cloth, capable of bending the belt cloth.

2. A belt according to claim 1, further comprising an inguinal region pressurization implement attached in the belt cloth, capable of breathing from the abdomen by pressurizing an inguinal region.

3. A belt bending attachment comprising:
a frame ornament;
an ornament engages with the frame ornament through the belt cloth, capable of bending the belt cloth, further comprises a root part, clip sticks formed at the both ends of the root part so as to project perpendicularly in a horizontal direction from both ends of the root part, a tip part thereof formed in shape of a crank, and a central clip stick provided so as to project perpendicularly from a central part of the root part, a tip part thereof formed in the shape of a crank having an opposite direction which differs from the direction of the tip part of the clip sticks.

4. A belt bending attachment according to claim 3, wherein the tip part of the central clip stick is formed straightly.
